(19) United States
(12) Patent Application Publication
(10) Pub. No.: US 2020/0245957 A1
(43) Pub. Date: Aug. 6, 2020

(54) RADIATION MEASUREMENT PENAL, DEVICE AND SYSTEM

(71) Applicant: YongLin Biotech Corp., New Taipei (TW)

(72) Inventors: LIANG-HSIANG WU, New Taipei (TW); NGOT-SWAN CHONG, New Taipei (TW); CHIH-CHING CHANG, New Taipei (TW); YI-CHING LIU, New Taipei (TW); MING-HSUN HSU, New Taipei (TW)

(21) Appl. No.: 16/262,955

(22) Filed: Jan. 31, 2019

(51) Int. Cl. A61B 6/00; A61N 5/10; G01T 7/00

(52) U.S. Cl. CPC A61B 6/4208; A61N 5/1071; A61N 2005/1092; G01T 7/00; A61B 6/4266

(57) ABSTRACT

A radiation measurement panel is disclosed. The radiation measurement panel comprising a substrate, a first conductive layer, a sacrificial layer, and a second conductive layer. The first conductive layer formed over the substrate. The sacrificial layer formed over the first conductive layer, wherein the dielectric constant of the sacrificial layer changes in accordance with a magnitude of received radiation. The second conductive layer formed over the sacrificial layer, wherein the magnitude of the received radiation corresponds to a capacitance between the first conductive layer and the second conductive layer.

### FIELD

The present disclosure generally relates to device and system for measuring radiation, and more particularly relates to device and system for measuring radiation used in radiotherapy.

### BACKGROUND

Radiation therapy may be curative in a number of types of cancer if they are localized to one area of the body. For example, radiotherapy is often performed for various types of cancers such as gastric cancer, colorectal cancer, pancreatic cancer, head and neck cancer, esophageal cancer, lung cancer, and breast cancer.

In one scenario, before the radiotherapy is performed, a doctor may decide a radiation dosage that should be absorbed by a target region of a human body that requires treatment based on a desired effect and the nature of the target region. Being able to accurately estimate an absorbed radiation dosage by the target region may help to track the outcome of the treatment.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates an exemplary operating environment of a radiation measurement device in accordance with some embodiments of the instant disclosure.

FIG. 2 illustrates a component block diagram of a radiation measurement device in accordance with some embodiments of the instant disclosure.

FIG. 3 shows a schematic sectional view of a radiation measurement panel in accordance with some embodiments of the instant disclosure.

FIG. 4 shows a schematic sectional view of a radiation measurement panel in accordance with some embodiments of the instant disclosure.

FIG. 5 illustrates an exemplary operating environment of a radiation measurement system in accordance with some embodiments of the instant disclosure.

It is to be noted, however, that the appended drawings illustrate only exemplary embodiments of this disclosure and are therefore not to be considered limiting of its scope, for the disclosure may admit to other equally effective embodiments.

### DETAILED DESCRIPTION

The present disclosure will now be described more fully hereinafter with reference to the accompanying drawings, in which exemplary embodiments of the disclosure are shown. This disclosure may, however, be embodied in many different forms and should not be construed as limited to the exemplary embodiments set forth herein. Rather, these exemplary embodiments are provided so that this disclosure will be thorough and complete, and will fully convey the scope of the disclosure to those skilled in the art. Like reference numerals refer to like elements throughout.

The terminology used herein is for the purpose of describing particular exemplary embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

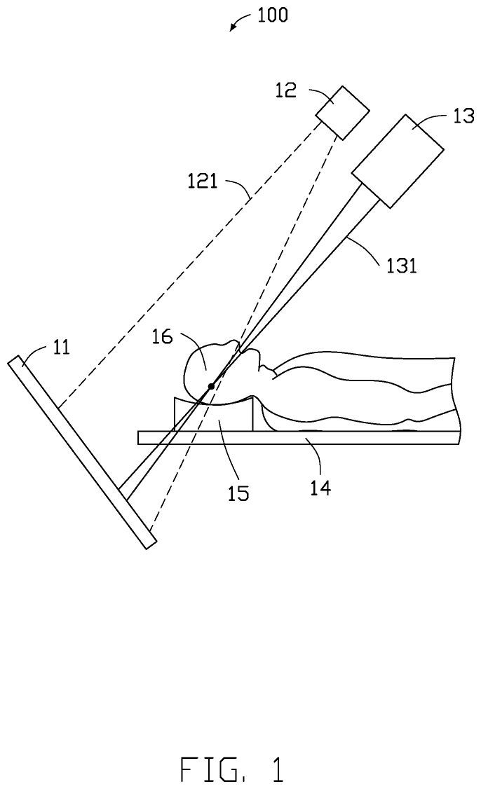

The description will be made as to the exemplary embodiments in conjunction with the accompanying drawings in FIG. 1 to 5. Reference will be made to the drawing figures to describe the present disclosure in detail, wherein depicted elements are not necessarily shown to scale and wherein like or similar elements are designated by same or similar reference numeral through the several views and same or similar terminology.

FIG. 1 illustrates an exemplary operating environment 100 of a radiation measurement device 11 in accordance with some embodiments of the instant disclosure.

The operating environment 100 comprises a radiation measurement device 11, a first radiation source 12, a second radiation source 13, a patient support 14 and a head support 15. In one scenario, the first radiation source 12 configured to emit first radiation 121. The first radiation 121 is used for generating medical image, and may include X-ray radiation. The second radiation source 13 is provided for emitting second radiation 131. The second radiation 131 can be used for cancer treatment, cell activity demodulation or tissue activity demodulation, and may include ionized radiation. In one scenario, the second radiation source 13 may include a Linear Accelerator.

In the illustrated scenario in FIG. 1, a patient is lying on the patient support 14, and the head of the patient is supported by the head support 15. The radiation measurement device 11 and the first radiation source 12 are arranged in a way that the first radiation 121 emitted from the first radiation source 12 penetrates the head portion of the patient and arrives at the radiation measurement device 11. Likewise, the second radiation source 13 is arranged in a way that the second radiation 131 emitted from the second radiation source 13 penetrates a target region 16 (e.g. the location of the tumor of the patient when the head of the patient is supported by the head support 15), and arrives at the radiation measurement device 11. In such a scenario, when the first radiation 121 and the second radiation 131 arrive at the radiation measurement device 11, among other features, the exemplary radiation measurement device 11 can perform a measurement/estimation of a dosage of the second radiation second radiation 131 irradiated on the radiation measurement device 11, and at the same time generate an internal image of the head of the patient.

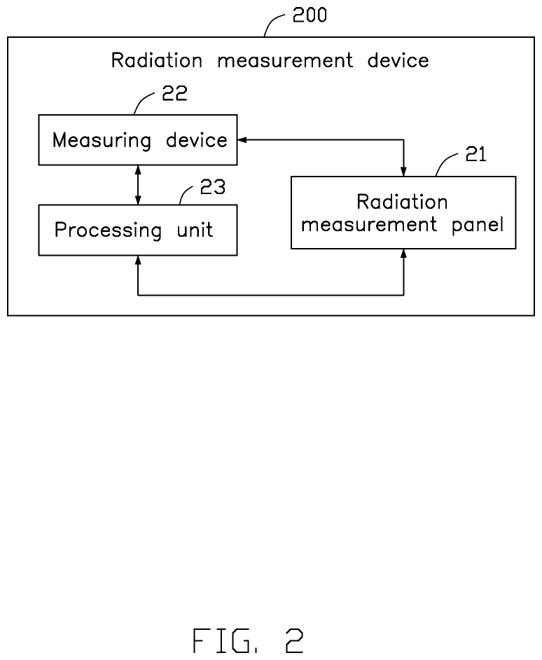

FIG. 2 illustrates an exemplary component block diagram of a radiation measurement device 200 in accordance with embodiments of the instant disclosure. The exemplary radiation measurement device 200 comprise a radiation measurement panel 21, a measuring device 22 coupled to the radiation measurement panel 21, and a processing unit 23 coupled to the radiation measurement panel 21 and the measuring device 22. To ensure flexible implementation, the radiation measurement panel 21 may be an integral component of the radiation measurement device 200, or a disposable product with one or more physical interface for establishing connections to the measuring device 22 and the processing unit 23.

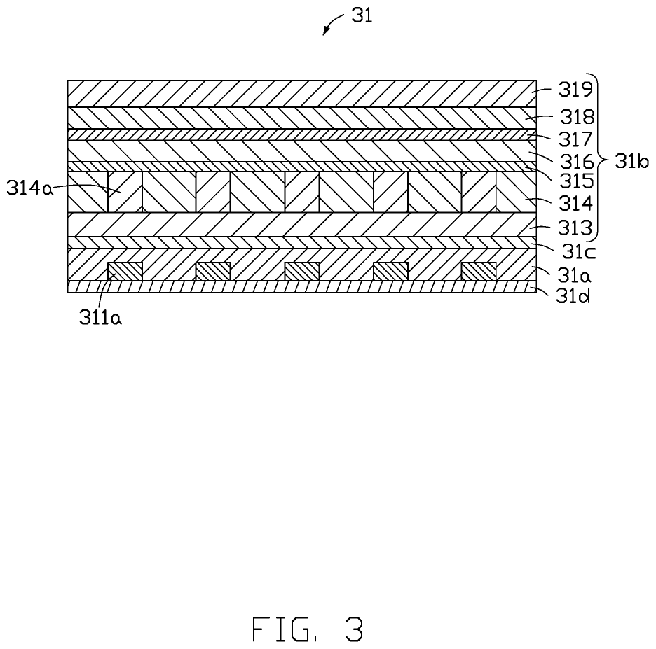

FIG. 3 illustrates an exemplary embodiment of the radiation measurement panel 31 of the instant disclosure. The exemplary radiation measurement panel 31 comprise a substrate 31d, a first subpanel 31a, a second subpanel 31b, and a third adhesive layer 31c.

The first subpanel 31a formed over the substrate 31d. The first subpanel 31a is configured to collect information for generating image data after receiving first radiation. In some embodiment, the first subpanel 31 may include an X-ray detecting flat panel. For example, the first subpanel 31 may comprise two-dimensionally arrayed X-ray detection pixels using thin-film transistors as a switching element, each pixel may include but not limited to a thin-film transistor 311a made of amorphous silicon (to be referred to as a-Si hereinafter) and used as a switching element, a conversion film made of Se, a pixel capacitor (to be referred to as a Cst hereinafter). When first subpanel 31 is irradiated by X-rays, charges are generated in the conversion film and stored in the Cst. When a scanning circuit turns on the thin-film transistors 311a, the stored charges which represents two-dimensional X-ray image can be read out by a circuit.

The third adhesive layer 312 formed on the first subpanel 31.

The second subpanel 31b includes a substrate 313, a first conductive layer 314, a first adhesive layer 315, a sacrificial layer 316, a second adhesive layer 317, a second conductive layer 318, and a cover layer 319.

The substrate 313 formed over the first subpanel 31 and is adhered with the first subpanel 31 by the third adhesive layer 312. The substrate 313 may include a combination of glass and plastics.

The first conductive layer 314 formed over the substrate 313. In the exemplary embodiment, the first conductive layer 314 comprises a plurality of discrete electrodes 314a. In some embodiment, the first conductive layer 314 itself may be a single electrode. In the exemplary embodiment, the first conductive layer 314 may be a transparent conductive film such as Indium Tin Oxide (ITO) film.

The first adhesive layer 315 is formed between the sacrificial layer 316 and the first conductive layer 314.

The sacrificial layer 316 generally includes a material whose dielectric properties degrades/changes with the exposure to the second radiation 131. The change of the dielectric property (e.g., dielectric constant) is measurable within the exposure time (e.g., instantaneous change is better but integrated measurement will work as well). In some embodiments. the sacrificial layer 316 is formed over the first conductive layer 314, wherein a dielectric constant of the sacrificial layer 316 changes in accordance with a magnitude of received second radiation 131. In the exemplary embodiment, a thickness of the sacrificial layer 316 ranges from about 0.1 μm to 10 μm. In the exemplary embodiment, the sacrificial layer 316 include a combination of Polyvinyl Chloride (PVC) and Polyethylene Terephthalate (PET).

The second adhesive layer 317 is formed between the sacrificial layer and the second conductive layer.

The second conductive layer 318 formed over the sacrificial layer 316, wherein The magnitude of the received second radiation 131 corresponds to a capacitance between the first conductive layer 314 and the second conductive layer 318. In the exemplary embodiment. the second conductive layer 318 is a transparent conductive film such as ITO film.

The measuring device 22 may include one or more hardware components, data or software applications for performing one or more functions described herein. For instance, the measuring device 22 may include one or more computer processors such as MCU, storing module as well as a variety of circuits as well as a table stored in the storing module. In one scenario, the table may describe relations between a plurality of dielectric constant and a plurality of reference radiation magnitude. In one scenario, the table may describe relations between a plurality of capacitance and a plurality of reference radiation magnitude.

By ways of example, in the exemplary embodiment, the measuring device 22 is configured to measure a dielectric constant change for estimating a magnitude of received second radiation 131 when a radiation measurement panel (e.g., the radiation measurement panel 31) is expose to the second radiation 131. In one scenario, the measuring device 22 is coupled to the first conductive layer 314 and the second conductive layer 318 of the radiation measurement panel 31 which is exposed to the second radiation 131, and configured to measure a capacitance between the first conductive layer 314 and the second conductive layer 318 for estimating the dielectric constant change and then estimating a magnitude of received second radiation 131 based on the measured capacitance and the table.

In one scenario, the measuring device 22 may estimate the change of dielectric constant (Δε) of the sacrificial layer 316 by measuring the capacitance between, the first conductive layer 314 and the second conductive layer 318 by applying the following formula:

C=(ε+Δε)A/d.

wherein C is the capacitance between the first conductive layer 314 and the second conductive layer 318, A is the area of the first conductive layer 314 and the second conductive layer 318, d is the thickness of the sacrificial layer 316.

In a scenario, the measuring device 22 may include a measuring circuit coupled to the first conductive layer 314 and the second conductive layer 316 for measuring a capacitance between the first conductive layer 314 and the second conductive layer 316. For example, the measuring circuit may be configured to apply one or more alternating currents to the first conductive layer 314 or the second conductive layer 318 to obtain one or more characteristic impedance for calculating the capacitance. In another example, the measuring circuit may comprise a resistor-capacitor (RC) circuit and MCU and be configured to calculate the capacitance using the time for charging and the time for discharging.

The processing unit 23 may include one or more hardware components, data or software applications for performing one or more functions described herein. For instance, the processing unit 23 may include one or more computer processors such as CPU, MCU, GPU, control chip, storing module as well as a variety of circuits as well as some information associated with the treatment stored in the storing module. The information associated with the treatment may include, but not limited to, a dosage of the second radiation 131 emitted from the second radiation source 13.

In the exemplary embodiment, the processing unit 23 is configured to estimate a radiation dosage absorbed by a patient under the second radiation 131 based on the estimated magnitude of received second radiation 131 and the dosage of the second radiation 131 emitted from the second radiation source 13 stored in the storing module. In one scenario, the radiation dosage absorbed by a patient under the radiation 131 plus the estimated magnitude of received second radiation 131 equals the dosage of the second radiation 131 emitted from the second radiation source 13.

In some embodiments, the radiation dosage absorbed by a patient under the radiation 131 may be used for future reference. For example, the radiation dosage absorbed by a patient may be displayed by a screen in data connection with the processing unit 23 for the purpose of providing essential information to a doctor who is in charge of the treatment for determining details of the next treatment (e.g., the direction and the dose of a radiation 131). In some embodiment, the processing unit 23 is further configured to transmit a dosage information to the second radiation source 13, the dosage information may indicate the radiation dosage absorbed by the patient. In some embodiment, the second radiation source 13 may store history information such as the dose and the direction of the second radiation 131 emitted from the second radiation source 13 as well as the dosage absorbed by the patient described in the dosage information from the processing unit 23. The history information may be considered as reference when setting some parameters (e.g., the dose and the direction of the second radiation 131) of the following treatment.

In some embodiment, the processing unit 23 is further configured to determine whether the dosage absorbed by a patient is larger than a predetermined value, and transmit a control signal for terminating the second radiation source 13 to the second radiation source 13.

In this embodiment, the processing unit 23 is further configured to generate an image based on the information collected by the first subpanel 31. In this embodiment, the processing unit 23 may include a scanning circuit configured to turn on the thin-film transistors 311a in the first subpanel 31, and a reading circuit configured to read the charges represents information for generating image data stored in the first subpanel 31 so as to generate an image.

In some embodiment, the processing unit 23 may further be provided with capability to analyze a movement (e.g., displacement, rotation, etc.) of the head of a patient by comparing the generated image and a reference image indicating an initial position of the head which had been set based on the position of the target region 15 relative to the second radiation 131, so as to provide a reference for determining whether to terminate the treatment. For instance, parameters describing the movement (e.g., magnitudes of displacement, rotation, etc.) of the head a may be displayed by a screen in data connection with the processing unit 23 for the purpose of providing a reference to a doctor who is in charge of the treatment for determining whether to terminate the treatment.

When the first conductive layer 314 comprises a plurality of discrete electrodes 314a, many values of capacitance between the electrodes 314a of the first conductive layer 314 and the second conductive layer 318 can be measured by the measuring device 22 such that many dielectric constant changes can be obtained. In such scenario, the measuring device 22 may further be configured to estimate a magnitude of received radiation 131 when the radiation measurement panel 31 is exposed to the second radiation 131 based on the largest one of measured dielectric constant changes. In such scenario, the processing unit 23 may further be configured to determine the accuracy of the second radiation 131 based on the dielectric constant changes corresponding to the electrodes 314a and the positions of the electrodes 314a.

It is noted that to treat target region 16 within the body of the patient, however, the second radiation 131 must typically penetrate healthy tissue in order to irradiate the target region 16. In conventional radiotherapy, large volumes of healthy tissue can thus be exposed to harmful dosage of the second radiation 131. Radiotherapy treatment plans are often constructed to achieve the desired on-site exposure whilst keeping the exposure of healthy cells to a minimum. in other word, when the movement of the patient is so large that increase the size of healthy tissue exposed to harmful dosage of the second radiation 131, the treatment must be terminated.

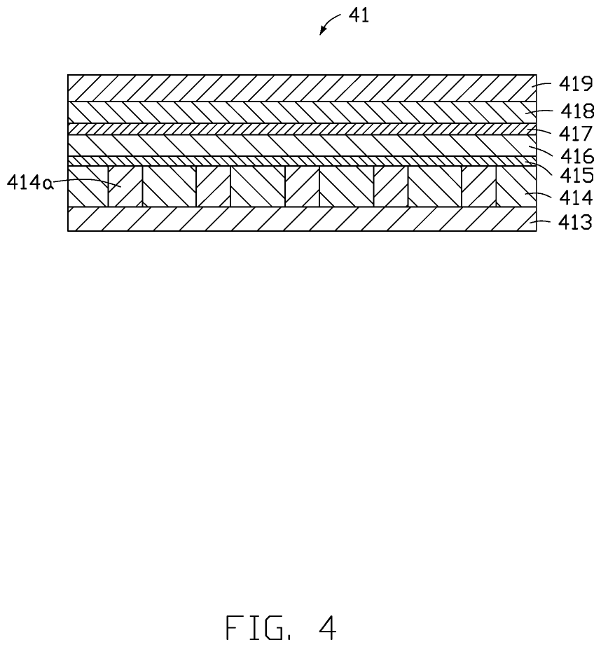

FIG. 4 illustrates an exemplary embodiment of the radiation measurement panel 41 of the instant disclosure. The exemplary radiation measurement panel 41 comprises a substrate 413, a first conductive layer 414, a first adhesive layer 415, a sacrificial layer 416, a second adhesive layer 417, a second conductive layer 418, and a cover layer 419.

The substrate 413 may include a combination of glass and plastics.

The first conductive layer 414 formed over the substrate 413. In some embodiment, the first conductive layer 414 comprises a plurality of discrete electrodes 414a. In other embodiment, the first conductive layer 414 itself may be a single electrode. In some embodiment, the first conductive layer 414 may be a transparent conductive film such as Indium Tin Oxide (ITO) film.

The first adhesive layer 415 is formed between the sacrificial layer 416 and the first conductive layer 414.

The sacrificial layer 416 is formed over the first conductive layer 414, wherein a dielectric constant of the sacrificial layer 414 changes in accordance with a magnitude of received radiation. The magnitude of the received radiation corresponds to a capacitance between the first conductive layer 414 and the second conductive layer 418. In some embodiment, a thickness of the sacrificial layer 416 ranges from about 0.1 μm to 10 μm. In some embodiment, the sacrificial layer 416 may include a combination of Polyvinyl Chloride (PVC) and Polyethylene Terephthalate (PET).

The second adhesive layer 417 is formed between the sacrificial layer 416 and the second conductive layer 418.

The second conductive layer 417 is formed over the sacrificial layer 416, wherein the magnitude of the received second radiation corresponds to a capacitance between the first conductive layer 414 and the second conductive layer 417. In some embodiment, the second conductive layer 417 is a transparent conductive film such as ITO film.

A dielectric constant change for estimating a magnitude of received radiation when the radiation measurement panel 41 is exposed to the radiation can be measured by a measuring device (e.g., the measuring device 22). In one scenario, a measuring device (e.g., the measuring device 22) is coupled to the first conductive layer 414 and the second conductive layer 418, and configured to measure a capacitance between the first conductive layer 414 and the second conductive layer 418 for estimating the dielectric constant change and then estimating a magnitude of received radiation based on the measured capacitance and the table.

In this embodiment, the measuring device (e.g., the measuring device 22) may include a measuring circuit coupled to the first conductive layer 414 and the second conductive layer 416 for measuring a capacitance between the first conductive layer 414 and the second conductive layer 416. For example, the measuring circuit may be configured to apply one or more alternating currents to the first conductive layer 414 or the second conductive layer 418 to obtain one or more characteristic impedance for calculating the capacitance. In another example, the measuring circuit may comprise a resistor-capacitor (RC) circuit and MCU and be configured to calculate the capacitance using the time for charging and the time for discharging.

In one scenario, the radiation measurement panel 41 may be disposed on a path of second radiation emitted from a second radiation source between a target region and the second radiation source, so as to receive the second radiation emitted from the second radiation source directly. In such scenario, the measurement device may perform a measurement/estimation of a dosage of the second radiation emitted from the radiation source.

In some embodiments, a processing unit (e.g., the processing unit 23) is configured to estimate a radiation dosage absorbed by a patient under the radiation based on the estimated magnitude of received radiation and the dosage of the radiation emitted from the radiation source stored in the storing module. In one scenario, the dosage of the radiation absorption by a patient under the radiation plus the estimated magnitude of received, radiation equals the dosage of the radiation emitted from the radiation source.

In some embodiments, the radiation dosage absorbed by a patient under the radiation may be used for future reference. For example, the radiation dosage absorbed by a patient may be displayed by a screen in data connection with a processing unit (e.g., the processing unit 23) for the purpose of providing essential information to a doctor who is in charge of the treatment for determine details of the next treatment (e.g., the direction and the dose of a radiation). When the first conductive layer 414 comprises a plurality of discrete electrodes 414a, many values of capacitance between the electrodes 414a of the first conductive layer 414 and the second conductive layer 418 can be measured by a measuring device (the measuring device 22) such that many dielectric constant changes can be obtained. In such scenario, the measuring device (e.g., the measuring device 22) may further be configured to estimate a magnitude of received radiation when the radiation, measurement panel 41 is exposed to the radiation based on the largest one of measured dielectric constant changes. In such scenario, the processing unit (e.g., the processing unit 23) may further be configured to determine the accuracy of the radiation based on the dielectric constant changes corresponding to the electrodes 414a and the positions of the electrodes 414a.

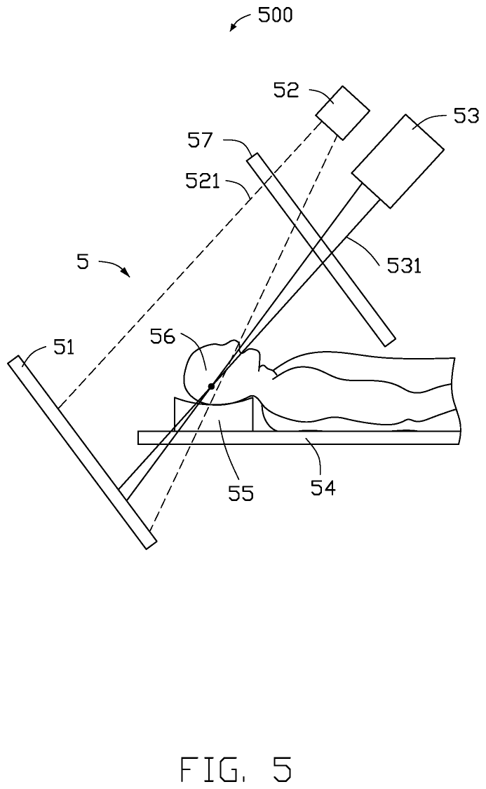

FIG. 5 illustrates an exemplary operating environment 500 of a radiation measurement system 5 in accordance with some embodiments of the instant disclosure.

The operating environment 500 comprises a radiation measurement system 5, a radiation source 53, a patient support 54 and a head support 55. In one scenario, the first radiation source 52 configured to emit first radiation 521. The first radiation 521 is used for generating medical image, and may include X-ray radiation. The second radiation source 53 is provided for emitting second radiation 531. The second radiation 531 can be used for cancer treatment; cell activity demodulation or tissue activity demodulation, and may include ionized radiation. In one scenario, the second radiation source 53 may include a Linear Accelerator.

In the illustrated scenario in FIGS, a patient is lying on the patient support 54, and the head of the patient is supported by the head support 55.

The radiation measurement system 5 comprises a first radiation measurement device 57 and a second radiation measurement device 51.

In one scenario, the first radiation measurement device 57 may comprise a radiation measurement panel and a measuring device. The radiation measurement panel comprises a substrate (e.g. substrate 413), a first conductive layer (e.g. first conductive layer 414), a sacrificial layer (e.g. sacrificial layer 414), a second conductive layer (e.g. second conductive layer 418). The a measuring device (e.g. measuring device 22) coupled to the radiation measurement panel, configured to measure a dielectric constant change for estimating a magnitude of received second radiation 531 when the radiation measurement panel is exposed to the second radiation 531.

The second radiation measurement device 51 comprising a radiation measurement panel, a measuring device and a processing unit. The radiation measurement panel of the second radiation measurement device 51 comprises a first subpanel and a second subpanel. The first subpanel (e.g. first subpanel 31a) is configured to collect information for generating image data after receiving first radiation 521. The second subpanel includes a substrate (e.g. substrate 313) formed over the first subpanel, a first conductive layer (e.g. first conductive layer 314) formed over the substrate, a sacrificial layer (e.g. sacrificial layer 316) formed over the first conductive layer, a second conductive layer (e.g. second conductive layer 318) formed over the sacrificial layer. The measuring device of the second radiation measurement device 51 coupled to the second subpanel, configured to measure a dielectric constant change for estimating a magnitude of received second radiation when the radiation measurement panel is exposed to the second radiation. The processing unit of the second radiation measurement device 51 couple to the first subpanel, configured to generate an image based on the information collected by the first subpanel.

The first and the second radiation measurement device 57,51 are configured to be arranged in a way that the second radiation 531 emitted from the second radiation source 53 passing sequentially through the first radiation measurement device 57, the target region 56, and arriving at the second radiation measurement device 51. In such a scenario, when the second radiation 531 arrive at the first radiation measurement device 57, among other features. the first radiation measurement device 57 can perform a measurement/estimation of a dosage of the radiation 531 emitted from the radiation source 53, and at the same time the second radiation measurement device 51 can perform a measurement/estimation of a dosage of the radiation 531 irradiated on the patient and generating a medical image of the patient.

In some embodiment, the second radiation source 53 measures the magnitude of the second radiation 531 with a dual ion chamber placed between a primary and a secondary collimator which does not provide a measurement of the radiation beam exiting the collimator or MLC (multi leaf collimator). This maybe the concept of monitor unit (MU). Some calibration are performed on daily/weekly basis to provide the measurement later. This means that this calibration has to be done on each system and is very critical for the treatment and it is a time consuming and costly task. In such embodiment the measurement/estimation of a dosage of the radiation 531 emitted from the radiation source 53 provided by the exemplary radiation measurement device 51′ may provide the measurement of the radiation beam exiting the collimator or MLC.

The embodiments shown and described above are only examples. Many details are often found in the art such as the other features of a radiation measurement panel and device. Therefore, many such details are neither shown nor described. Even though numerous characteristics and advantages of the present technology have been set forth in the foregoing description, together with details of the structure and function, the disclosure is illustrative only, and changes may be made in the detail, especially in matters of shape, size, and arrangement of the parts within the principles , up to and including the full extent established by the broad general meaning of the terms used in the claims. It will therefore be appreciated that the embodiments described above may be modified within the scope of the claims.

What is claimed is:

1. A radiation measurement panel, comprising:
a substrate;
a first conductive layer formed over the substrate;
a sacrificial layer formed over the first conductive layer, wherein a dielectric constant of the sacrificial layer changes in accordance with a magnitude of received radiation; and
a second conductive layer formed over the sacrificial layer, wherein the magnitude of the received radiation corresponds to a capacitance between the first conductive layer and the second conductive layer.

2. The panel as claimed in claim 1, wherein the second conductive layer is a transparent conductive film.

3. The panel as claimed in claim 1, wherein the first conductive layer comprises a plurality of discrete electrodes.

4. The panel as claimed in claim 1, a thickness of the sacrificial layer ranges from about 0.1 μm to 10 μm.

5. The panel as claimed in claim 1, wherein the sacrificial layer includes a combination of Polyvinyl Chloride (PVC) and polyethylene terephthalate (PET).

6. The panel as claimed in claim 5, further comprising:
a first adhesive layer formed between the sacrificial layer and the first conductive layer; and
a second adhesive layer formed between the sacrificial layer and the second conductive layer.

7. A radiation measurement device, comprising:
a radiation measurement panel comprising:
a substrate;
a first conductive layer formed over the substrate,
a sacrificial layer formed over the first conductive layer, wherein the dielectric constant changes in accordance with a dosage of received radiation; and
a second conductive layer formed over the sacrificial layer and in contact with the sacrificial layer, wherein the magnitude of the received radiation corresponds to a capacitance between the first conductive layer and the second conductive layer; and
a measuring device coupled to the radiation measurement panel, and configured to measure a dielectric constant change for estimating a magnitude of received radiation when the radiation measurement panel is exposed to the radiation.

8. The device as claimed in claim 7, wherein the measuring device is coupled to the first conductive layer and the second conductive layer, and configured to measure a capacitance between the first conductive layer and the second conductive layer for estimating the dielectric constant change.

9. The device as claimed in claim 7, further comprising a table describing relations between a plurality of dielectric constant and a plurality of reference radiation magnitude.

10. The device as claimed in claim 7, further comprising a table describing relations between a plurality of capacitance and a plurality of reference radiation magnitude.

11. The device as claimed in claim 7, further comprising a processing unit configured to estimate a radiation dosage absorbed by a patient under radiation based on the estimated magnitude of received radiation.

12. The device as claimed in claim 7,
wherein, the first conductive layer comprises a plurality of discrete electrodes; and
wherein, the measuring device is further configured to estimate a magnitude of received radiation when the radiation measurement panel is exposed to the radiation based on the largest one of measured dielectric constant changes.

13. A radiation measurement panel, comprising:
a first subpanel, configured to collect information for generating image data upon receiving first radiation;
a second subpanel, including:
a substrate formed over the first subpanel;
a first conductive layer formed over the substrate;
a sacrificial layer formed over the first conductive layer, wherein a change in dielectric constant of the sacrificial layer corresponds to a magnitude of received second radiation; and
a second conductive layer formed over the sacrificial layer, wherein the magnitude of the received second radiation corresponds to a capacitance between the first conductive layer and the second conductive layer.

14. The panel as claimed in claim 13, wherein the second conductive layer is a transparent conductive film.

15. The panel as claimed in claim 13, wherein the first conductive layer comprises a plurality of discrete electrodes.

16. The panel as claimed in claim 13, a thickness of the sacrificial layer ranges from about 0.1 μm to 10 μm.

17. The panel as claimed in claim 13, wherein the sacrificial layer includes a combination of Polyvinyl Chloride (PVC) and Polyethylene Terephthalate (PET).

18. The panel as claimed in claim 17, further comprising:
a first adhesive layer formed between the sacrificial layer and the first conductive layer; and
a second adhesive layer formed between the sacrificial layer and the second conductive layer.

19. The panel as claimed in claim 13, further comprising a third adhesive layer locates between the first subpanel and the substrate of the second subpanel.

20. A radiation measurement device, comprising:
a radiation measurement panel comprising:
a first subpanel, configured to collect information for generating image data after receiving first radiation;
a second subpanel, including:
a substrate formed over the first subpanel;
a first conductive layer formed over the substrate;
a sacrificial layer formed over the first conductive layer, wherein, a change in dielectric constant of the sacrificial layer corresponds to a magnitude of received second radiation; and
a second conductive layer formed over the sacrificial layer, wherein the magnitude of the received second radiation corresponds to a capacitance between the first conductive layer and the second conductive layer;
a measuring device coupled to the second subpanel, configured to measure a dielectric constant change for estimating a magnitude of received second radiation when the radiation measurement panel is exposed to the second radiation; and
a processing unit couple to the first subpanel, configured to generate an image based on the information collected by the first subpanel.

21. The device as claimed in claim 20, wherein the measuring device is coupled to the first conductive layer and the second conductive layer. and configured to measure a capacitance between the first conductive layer and the second conductive layer for estimating the dielectric constant change.

22. The device as claimed in claim 20, further comprising a table describing relations between a plurality of dielectric constant and a plurality of reference radiation magnitude.

23. The device as claimed in claim 20, the processing unit is further configured to estimate a radiation dosage absorbed by a patient under the second radiation based on the estimated magnitude of received second radiation.

24. The device as claimed in claim 20,
wherein, the first conductive layer comprises a plurality of discrete electrodes; and
wherein, the measuring device is further configured to estimate a magnitude of received second radiation when the radiation measurement panel is exposed to the second radiation based on the largest one of measured dielectric constant changes.

25. A radiation measurement system, adapted to be arranged in a path of a second radiation from a second radiation source, the system comprising:
a first radiation measurement device, arranged between the second radiation source and a target region, comprising:
a radiation measurement panel comprising:
a substrate;
a first conductive layer formed over the substrate;
a sacrificial layer formed over the first conductive layer, wherein a change in dielectric constant of the sacrificial layer corresponds to a magnitude of received second radiation; and
a second conductive layer formed over the sacrificial layer, wherein the magnitude of the received second radiation corresponds to a capacitance between the first conductive layer and the second conductive layer;
a measuring device coupled to the radiation measurement panel, configured to measure a dielectric constant change for estimating a magnitude of received second radiation when the radiation measurement panel is exposed to the second radiation.

26. The system as claimed in claim 25, further comprising:
a second radiation measurement device, comprising:
a radiation measurement panel comprising:
a first subpanel, configured to collect information for generating image data after receiving first radiation;
a second subpanel, including:
a substrate formed over the first subpanel:
a first conductive layer formed over the substrate;
a sacrificial layer formed over the first conductive layer, wherein a change in dielectric constant of the sacrificial layer corresponds to a magnitude of received second radiation; and
a second conductive layer formed over the sacrificial layer, wherein the magnitude of the received second radiation corresponds to a capacitance between the first conductive layer and the second conductive layer;
a measuring device coupled to the second subpanel, configured to measure a dielectric constant change for estimating a magnitude of received second radiation when the radiation measurement panel is exposed to the second radiation; and
a processing unit couple to the first subpanel, configured to generate an image based on the information collected by the first subpanel.

27. The system as claimed in claim 26, wherein the first and the second radiation measurement device are configured to be arranged in a way that the second radiation emitted from the second radiation source passing sequentially through the first radiation measurement device, the target region, and arriving at the second radiation measurement device.
